(19) United States
(12) Patent Application Publication
(10) Pub. No.: US 2020/0037607 A1
(43) Pub. Date: Feb. 6, 2020

(54) Materials and Methods for Delivering a Substance to a Plant

(71) Applicant: University of Florida Research Foundation, Incorporated, Gainesville, FL (US)

(72) Inventors: Luis V. Ponce Cabrera, Gainesville, FL (US); ED Etxeberria, Gainesville, FL (US)

(21) Appl. No.: 16/529,138

(22) Filed: Aug. 1, 2019

Related U.S. Application Data
(60) Provisional application No. 62/715,002, filed on Aug. 6, 2018.

(51) Int. Cl. A01N 25/04; A01N 3/00; A01G 7/04; A01N 25/24

(52) U.S. Cl. CPC A01N 25/04; A01N 3/00; C12N 15/8207; A01N 25/24; A01G 7/045

(57) ABSTRACT

The present disclosure is directed to materials and methods for delivering substances to plants using light energy.

### CROSS-REFERENCE TO RELATED APPLICATION

The present application claims the benefit of priority to U.S. Provisional Application No. 62/725,002, filed Aug. 6, 2018, the disclosure of which is incorporated herein by reference in its entirety.

### FIELD OF THE INVENTION

The present disclosure is directed to materials and methods for delivering substances to plants.

### BACKGROUND

Application and absorption of agrichemicals (e.g., foliar nutrients and pesticides) remains difficult in the agricultural field. Leaves are the ideal target, as they are the most readily accessible tissue and represent a significant proportion of the total plant body. In leaves, stomatal openings provide a means for movement of substances across the cuticle and into leaves. Stomata significantly contribute to the overall exchange of hydrophilic substances across leaf surfaces; nevertheless, the collective area of stomatal openings that allow for penetration into the leaf is restricted, even under optimum circumstances, because (1) stomata often close under a variety of biotic and abiotic conditions, (2) only a small percentage of the opened stomata participate in the uptake process, and (3) the entire stomata opening is not available to the movement of aqueous solutions because stomates are protected against infiltration by their geometry and pore walls. The effectiveness of aerial sprays, a preferred means of delivering agrichemicals over large areas, is limited because of inefficient transport of substances across the cuticular barrier that protects all leaves. There remains a need in the art for an effective method for delivering substances into plants on a large scale (i.e., in fields).

### SUMMARY OF THE INVENTION

The disclosure provides a method of delivering a substance to a plant, the method comprising (a) exfoliating a region of epicuticular wax on a plant surface and (b) applying the substance to the exfoliated region. Optionally, the plant is a tree, a vine, a forage, a perennial crop, a row crop, a bush crop, an ornamental plant, an annual plant, or a grass. In various embodiments, the plant is a citrus tree, such as Citrus x sinensis. In various aspects, the substance is an agrochemical. In various aspects, the substance is an exogenous nucleic acid. In some embodiments, the exogenous nucleic acid is selected from the group consisting of an antisense RNA (asRNA), a micro RNA (miRNA), a small interfering RNA (siRNA), a double-stranded RNA (dsRNA), a non-coding RNA (ncRNA), mitochondrial RNA (mtRNA), and combinations thereof, and/or the exogenous nucleic acid is not incorporated in a viral vector. In various aspects, the substance is a fertilizer, an insecticide, a fungicide, a microelement, or a plant hormone.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. The terms “comprising,” “having,” “including,” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities of ingredients or reaction conditions used herein should be understood as modified in all instances by the term “about” as that term would be interpreted by the person skilled in the relevant art.

The entire document is intended to be related as a unified disclosure, and it should be understood that all combinations of features described herein are contemplated, even if the combination of features are not found together in the same sentence, paragraph, or section of this document. All references cited herein are hereby incorporated by reference.

### BRIEF DESCRIPTION OF THE FIGURES

FIG. 1 is a schematic of an embodiment of the wax exfoliation concept.

FIGS. 2A and 2B are representations of epicuticular wax (FIG. 2A) before laser application and (FIG. 2B) during laser application, depicting laser-mediated exfoliation of the wax.

FIGS. 3A and 3B are images of citrus leaves taken two hours after fluorescent glucose application. FIG. 3A depicts a leaf which was not treated with a laser prior to glucose application; FIG. 3B depicts a laser-treated leaf.

FIGS. 4A and 4B are images of a leaf after laser treatment. FIG. 4A is a depiction of the surface; in FIG. 4B the surface has been tinted to highlight the contrast.

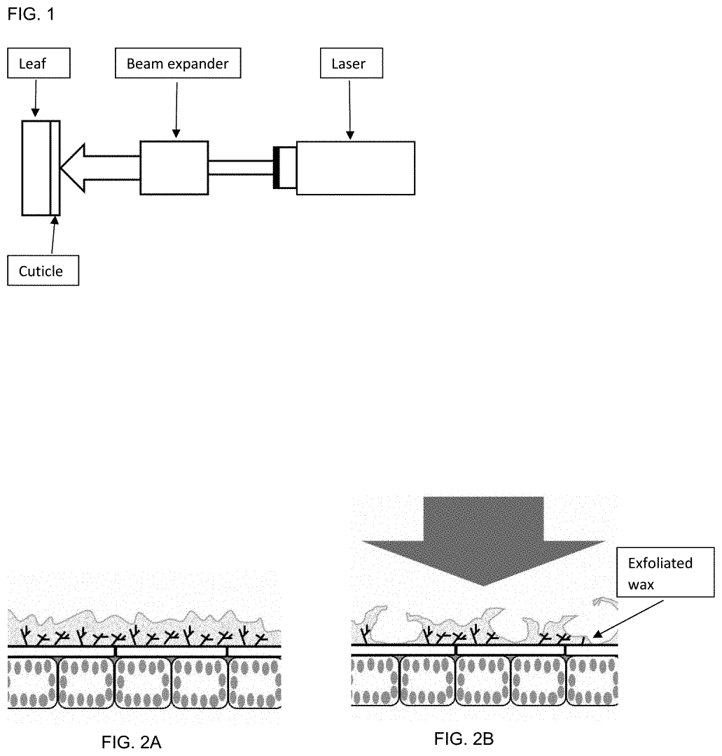

### DETAILED DESCRIPTION

The present disclosure provides materials and methods in which light energy can be used to enhance the penetration of a substance into plants. This is accomplished by applying a light energy to a plant surface (e.g., a leaf of the plant) to exfoliate the wax from the surface of the cuticle (i.e., epicuticular wax), without fully removing the wax and without damaging the leaf structure. No tissue perforations (e.g., pore, crater, or other indentation) occur in this method. Put another way, the epidermis of the plant is not punctured by the light energy (there is no physical damage the leaf structure). The instant method can be applied to more surface area of the plant compared to methods which require creating pores or holes in the tissue underlying the epicuticular wax (the epidermis and mesophyll), which can damage the plant and sometimes require application of a new layer of wax to prevent infection. In contrast, the wax layer of the plant heals itself after exfoliation. Further, the machinery required for methods that puncture underlying plant tissue is superbly expensive because of the small treated area per laser pulse, the complexity of instrumentation, and the problem of focalization. The treatment area of the instant method need not be so localized.

In one aspect, described herein is a method of delivering a substance to a plant, the method comprising (a) exfoliating a region of epicuticular wax on a plant surface and (b) applying the substance to the exfoliated region.

The delivery of any substance beneficial to the plant is specifically contemplated. In some embodiments, the substance is selected from the group consisting of an exogenous nucleic acid, a fertilizer, an insecticide, a fungicide, a microelement, zinc nanoparticles, and a plant hormone. Combinations of any of the foregoing are contemplated. Alternatively, the substance is harmful to the plant and applied to, for instance, kill or inhibit the growth or spread of unwanted plants (e.g., weeds).

In one aspect, the disclosure provides a method of delivering at least one exogenous nucleic acid to a plant. The nucleic acid may encode a product that imparts a benefit or provides a function in the plant. Alternatively, the nucleic acid, itself, may impart a function or provide a benefit in the plant. The exogenous nucleic acid may be DNA or RNA. Nucleic acid may comprise sequences that support replication and/or protein production. Optionally, the nucleic acid does not originate (i.e., is not derived from) a plant sequence; in this regard the nucleic acid sequence is optionally an animal, bacteria, insect, yeast, or fungi nucleic acid sequence. In various aspects, the exogenous nucleic acid is selected from the group consisting of an antisense RNA (asRNA), a micro RNA (miRNA), a small interfering RNA (siRNA), a double-stranded RNA (dsRNA), a non-coding RNA (ncRNA), mitochondrial RNA (mtRNA), and combinations thereof. For example, RNAi may be employed to, e.g., modify gene expression to increase resistance to a pathogen in the plant (e.g., interfere with the metabolism or development process, affect the development of an insect (e.g., Citrus psyllid) that feeds on the plant); suppress a virus-based vector in the plant, or modify gene expression to obtain a favorable trait in the plant. The nucleic acid may interact with native nucleic acids or with nucleic acids previously introduced into the plant (e.g., transgenes) to modulate expression.

In various aspects, the nucleic acid is not present in a viral vector. Virus-induced gene silencing (VIGS) takes advantage of the host plant's own natural defense mechanisms to suppress intruding viruses, which are themselves usually single strands of RNA. Viruses such as tobacco mosaic virus (Ruiz et al., Plant Cell 10, 937-946, 1998), potato virus X (Anandalakshmi et al., Proc. Natl. Acad. Sci. U.S.A 95, 13079-13084, 1998), and tobacco rattle virus (Ratcliff et al., Plant J. 25, 237-245, 2001) have been used with VIGS for delivery of endogenous plant genes. This method can take up to nine months for the desired phenotype to be observed. In addition, such methods of nucleic acid delivery can only be used in laboratory settings. Viral vectors are not required in the context of the disclosure, and the ability to efficiently deliver nucleic acid without the use of viral vectors is a further advantage, allowing large scale, field use.

Exemplary fertilizers include, but are not limited to, ammonium nitrate, Ammonium sulfate, anhydrous ammonia, calcium nitrate/urea, oxamide, potassium nitrate, urea, urea sulfate, ammoniated superphosphate, diammonium phosphate, nitric phosphate, potassium carbonate, potassium metaphosphate, calcium chloride, magnesium ammonium phosphate, magnesium sulfate, ammonium sulfate, and potassium sulfate. Exemplary insecticides include, but are not limited to, Aldicarb, Bendiocarb, Carbofuran, Ethienocarb, Fenobucarb, Oxamyl, Methomyl, Acetamiprid, Clothianidin, Dinotefuran, Imidacloprid, Nitenpyram, Nithiazine, Thiacloprid, Thiamethoxam, Mirex, Tetradifon, Phenthoate, Phorate, Pirimiphos-methyl, Quinalphos, Terbufos, Tribufos, Trichlorfon, Tralomethrin, Transfluthrin, Fenoxycarb, Fipronil, Hydramethylnon, Indoxacarb, and Limonene. Additional exemplary insecticides include Carbaryl, Propoxur, Endosulfan, Endrin, Heptachlor, Kepone, Lindane, Methoxychlor, Toxaphene, Parathion, Parathion-methyl, Phosalone, Phosmet, Phoxim, Temefos, Tebupirimfos, and Tetrachlorvinphos. Exemplary fungicides include, but are not limited to, azoxystrobin, cyazofamid, dimethirimol, fludioxonil, kresoxim-methyl, fosetyl-A1, triadimenol, tebuconazole, and flutolanil. Exemplary microelements include, but are not limited to, iron, manganese, boron, zinc, copper, molybdenum, chlorine, sodium, cobalt, silicon, and nickel. Exemplary hormones include, but are not limited to, auxins (such as heteroauxin and its analogues, indolylbutyric acid and a-naphthylacetic acid), gibberellins, cytokinins, strigolactones, melatonin, and abscisic acids. Combinations of any of the foregoing are contemplated.

Any plant is suitable for treatment in the context of the disclosed method. In some aspects, the plant is a higher plant. In various aspects, the plant is a tree, a vine, forage, a perennial crop, a row crop, a bush crop, an ornamental plant, an annual plant, or a grass. In various embodiments, the plant is a fruit tree selected from the group consisting of an apple tree, a peach tree, a citrus tree, an olive tree, a cherry tree, a pear tree, a plum tree, a grapefruit tree, and an apricot tree. In various embodiments, the plant is a citrus tree, such as Citrus x sinensis, Citrus macrophylla, Citrus assamensis, Citrus aurantiaca, Citrus aurantiifolia, Citrus aurantium L., Citrus australasica, Citrus australis, Citrus cavaleriei, Citrus garrawayi, Citrus glauca, Citrus gracilis, Citrus halimii, Citrus hystrix, Citrus indica, Citrus inodora, Citrus japonica, Citrus junos, Citrus khasya, Citrus latifolia, Citrus latipes, Citrus limon (L.), Citrus maxima (Burro.), Citrus medica L., Citrus paradise, Citrus polyandra, Citrus x polytrifolia, Citrus reticulate, Citrus swinglei, Citrus trifoliata L., Citrus warburgina, or Citrus wintersii.

In various aspects, the plant is infected with a pathogen or suffers from a disease, or is at risk of pathogen infection or disease. The substance preferably provides a therapeutic effect in the plant such as, but not limited to, curing the disease, reducing the symptoms of disease, reducing an infected area of the plant, reducing pathogen load, reducing symptoms of pathogen infection, and the like. The disease or pathogen may be localized (i.e., in one or more “infected sites”) or may be widespread (i.e., “systemic”) throughout the plant. An advantage of the instant method is the ability to treat systemic disease via topical application of the substance. Examples of diseases include, for example, Huanglongbing (HLB, also called citrus greening disease), Citrus Tristeza Virus (CTV), Citrus Variegated Chlorosis (CVC), Laurel wilt disease, Fusarium wilt, Phytoplasmas, Zebra chip disease, bacterial kiwifruit vine disease, Chestnut blight, Oak wilt, Fusarium wilt, and Pierce's disease.

In various aspects, the plant is a citrus tree suffering from HLB. The Florida citrus industry has been severely impacted by HLB, a disease stemming from the phloem-limited Candidatus Liberibacter asiaticus (CLas). All citrus varieties are susceptible to HLB, and disease-tolerant trees are few. HLB is spread by the Asian Citrus psyllid, Diaphorina citri, the vector insect of CLas. The bacterium multiplies in host trees and causes symptoms of blotchy mottle in the leaves, poor fruit quality, a high rate of fruit drop resulting in reduced yield, and eventual tree death. In various aspects, the disclosure provides a method of treating a citrus tree suffering from HLB, wherein the method comprises exfoliating epicuticular wax in a region of a leaf surface using light energy, and applying a composition that kills or inhibits the growth of Candidatus Liberibacter to the region of the leaf.

The plant need not be infected with a pathogen or suffering from disease; the disclosure also contemplates prevention of infection or otherwise enhancing the health or production of a plant using the method described herein. In these aspects, the plant is a healthy plant (i.e., not suffering for disease or infection). In this regard, the substance may impart a beneficial property to the plant such as increased yield, enhanced color or heartiness, or pest resistance.

In some embodiments, the plant surface is the target area of the plant where delivery of the substance is desired. For example, the surface of the plant may be affected by pathogen or disease, and the substance provides some benefit to the surface region. Alternatively, the target area may be distal from the surface region where the exfoliation and/or substance is applied. In this regard, the method described herein provides a means of delivering a substance to internal structures of the plant by topical application. For example, the surface region that is treated may be a surface of at least one of a leaf, a stem, and/or a branch, while the infected site in which a therapeutic effect is desired is in a phloem. Indeed, the disclosure contemplates that the region to which the composition is applied and the infected site or region in which the substance is desired are in distinct plant systems (e.g., dermis, vascular, ground). To illustrate, the region to which the composition is applied may be in the dermal tissue system while the infected site to be treated is in the vascular system. Alternatively, the surface region which is exfoliated and treated may be located on a first leaf, and the target region could be located on a second leaf, optionally located on different stems and/or branches.

In some aspects where the target area is distal from the surface region that is treated, the substance does not exert an effect at the surface region but does elicit a biological response after travelling through systemic pathway. This is not required, however; the substance may exert an effect at the surface region and, in various aspects, may remain localized at the surface region or adjacent tissue (e.g., within the same leaf, or within the leaves on a single branch). In some embodiments, the substance may be localized in the phloem adjacent to surface region and not travel to the roots. Optionally, at least 20%, at least 30%, at least 50%, at least 75%, or at least 90% of the substance is absorbed by the phloem and/or the xylem of the plant.

The method contemplates applying the light energy to the surface region prior to applying the substance, although the disclosure is not so limited. It is also contemplated that the substance may be applied to the plant, and the light energy subsequently applied to exfoliate the surface region to enhance uptake of the substance. The application of light energy and the application of the substance is performed sufficiently close in time to allow uptake of the substance. For example, application of the light energy and application of the substance may occur within less than two hours, e.g., less than one hour, less than 30 minutes, less than 10 minutes, less than five minutes, or within 1 minute of each another. In various aspects, the substance is applied immediately after application of the light energy.

The method of the disclosure comprises exfoliating the epicuticular wax on a plant surface by light energy. By “exfoliating the epicuticular wax on a plant surface” is meant introducing a series of cracks or disruptions of the epicuticular wax on the surface of the plant. The cracks or disruptions in the wax do not extend through a surface region into an underlying region of the plant (e.g., do not extend to the epidermis or through various layers of the plant). In other words, the epidermis remains intact (i.e., with no perforations, cracks or disruptions).

Depending on the plant (e.g., leaf) surface and wax to be exfoliated, diverse laser types and laser wavelengths can be used, ranging from ultraviolet (UV) to infrared (IR). In the context of the method disclosed herein, focalizing the laser is unnecessary (although focusing the light energy to particular regions also is contemplated). A laser beam may be employed without any additional optical element taking advantage of its low angular divergence. A schematic of a representative embodiment is set forth in FIG. 1. This allows application of the light energy from different distances, irradiating leaves located in different parts of the plant, without the need to focalize the beam which, e.g., entails moving the laser source. Exfoliation is possible by simply orienting the laser toward the leaves, as if it were a light beam, in various aspects.

Several types of laser, as well as different parameters, can be used for exfoliation. The selection of the type of laser and the parameters depends on the optical properties of the leaves of the particular specie. Suitable lasers for use in the method include, but are not limited to, an erbium-doped yttrium aluminum garnet (Er:YAG) laser, Nd:YAG, Alexandrite laser, diode laser, fiber laser, and CO₂ laser. In various aspects, the laser is a solid state laser. Energy densities can be adjusted depending on the optical properties, composition and thickness of the wax layer, and the nature of the bottom substrate for different plants. Pulse repetition rates can be adjusted for efficiency of exfoliation and adequate control of the cleaning procedure by the operator. The wavelength employed preferably demonstrates a high absorption by water. Suitable wavelengths include, e.g., between 500 nanometers and 100 microns (e.g., 1-50 microns). Suitable pulse width is, in various aspects, between 1 nanosecond and 1 millisecond (e.g., between 1 nanosecond and 1 microsecond or between 1 microsecond and 1 millisecond). The repetition of pulses can be from a single pulse to 10 Kilohertz. Pulse energy employed may be, e.g., between 10 millijoules up to 10 joules.

In various aspects, the instant method does not comprise use of a focused laser beam, which generates holes in underlying plant tissue (i.e., creates incisions or ruptures in epidermis), requires use of a system of lenses, and has a limited working distance. A parallel laser beam is utilized in various aspects of the disclosure, which provides more flexibility as to instrumentation or apparatus, does not require constant focal adjustment, and allows light energy application from farther distances. In various aspects, the laser is located at least 0.5 meters from the plant, e.g., at least 1, at least 1.5, at least 2, at least 2.5, or at least 3 meters from the plant. Further, the laser fluence employed is below the ablation threshold, such that there is minimal damage to the epidermis.

In various aspects, light energy is applied using an erbium-doped yttrium aluminum garnet (Er:YAG) laser. In various aspects, suitable parameters for light energy include a wavelength having about 250-3000 nanometers. In some embodiments, the method comprises exposing the epicuticular wax on the plant surface (e.g., leaf) to random irradiation from a Er:YAG (erbium-doped yttrium aluminum garnet) laser characterized by a 2940 nm wavelength, a 200 μs pulse length, a constant repetition rate of 10 Hz, and an energy density of 280 mJ/cm² through a laser beam expander placed between the Er:YAG laser beam and the regions of a leaf surface. This arrangement ensures the energy density inside the laser spot by using parallel laser beams which avoid irradiating the leaves from any distance in the range between 0.5 m and 5 m. Other wavelengths may be employed including, but not limited to, 2940, 1064, 355, and 266 nm, as well as other pulse lengths, repetition rates, and energy densities. In the case of citrus, for example, representative parameters include, but are not limited to, pulse energy of 500 mJ, wavelength of 2940 nm of Er:YAG laser, pulse duration of 200 microseconds, and repetition of 100 Hz. The irradiation may be perpendicular to the substrate or under another angle.

In aspects of the disclosure wherein a substance (e.g., an agrochemical or nucleic acid) is applied following exfoliation, the composition comprising the substance may comprise additional components, such as one or more stabilizers, surfactants, degradation inhibitors (e.g., a DNAse inhibitor or an RNAse inhibitor), buffers, and/or antimicrobial agents.

Any method of applying the substance to the surface of the plant is suitable for use in the method so long as a sufficient amount of substance is applied to achieve a desired result (e.g., surface coverage). Suitable methods include, but are not limited to, spraying, dusting, sprinkling (e.g., via an irrigation system or release from a crop duster), brushing, smearing, and drenching. Application of the substance need not be limited to the precise surface region that is exfoliated, but the disclosure does also contemplate such targeted delivery.

The method of the disclosure may be performed multiple times to achieve a desired response in the plant. The level of exfoliation (e.g., amount of surface covered, amount of wax removed or disrupted) may be adjusted between applications, as well as the amount of the substance applied. Additionally, the substance may be applied more than once after the exfoliation step, and optionally one or more additional substances, which may be the same or different, are applied.

### Example

This Example demonstrates foliar uptake of a substance following epicuticular was exfoliation of the surface of leaves of a citrus plant using light energy.

The leaves of citrus tree (Citrus x sinensis) were exposed to an Er:YAG laser (2940 nm, 200 μs pulse length) in order to confirm its effect on the leaves wax. The samples were irradiated perpendicular to the surface in air. In all cases, one single pulse was enough to obtain the effect of wax exfoliation. The laser energy fluence employed for the removal of wax over leaves ranged from 0.3-3.2 J/cm² depending on the laser energy level.

As a method for visual checking of substance penetration into the leaf structure, a fluorescent glucose solution was added to the leaf surface.

Microscopic observations were made using a Carl Zeiss Axion Scope A-1 equipped with a Canon EOS Rebel T3i camera and a Carl Zeiss AxioCam ICc 1. Low magnification images were taken with a Zeiss Stemi SV11 fluorescent stereoscope (Carl Zeiss Microscopy GmbH, Gottingen, Germany) five minutes after the fluorescent glucose composition was applied to the leaves.

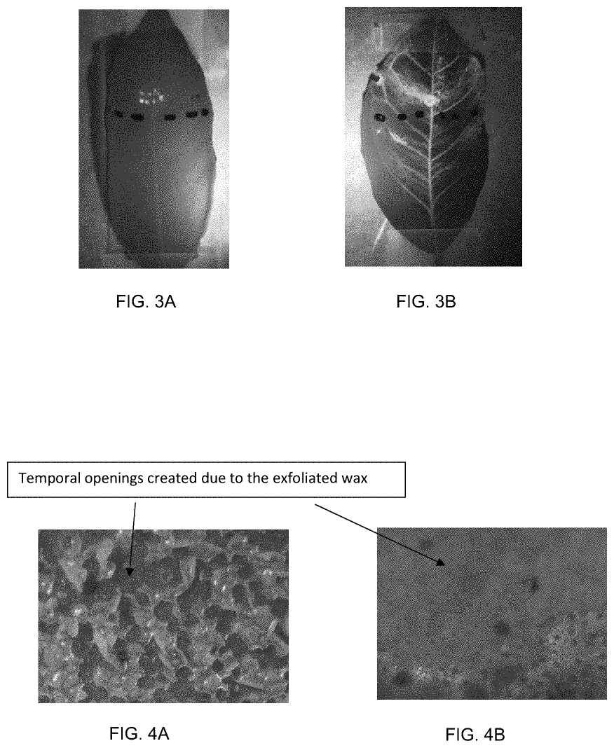

As shown in FIG. 3A, the fluorescent glucose solution was unable to penetrate the epicuticular wax in a leaf that was not treated with the laser; the solution did not spread throughout the control leaf. In contrast, as shown in FIG. 3B, the same quantity of solution penetrated and spread throughout the exfoliation-treated leaf.

FIGS. 4A and 4B confirm that the laser treatment did not damage the treated leaves. Images of both leaf surface and cross-section after laser treatment are presented. FIG. 4A shows the exfoliated areas free of wax and that cellular structure is preserved. The cross-section image in FIG. 4B shows no crater or damage to the laser-treated leaf.

1. A method of delivering a substance to a plant, the method comprising (a) exfoliating a region of epicuticular wax on a plant surface and (b) applying the substance to the exfoliated region.

2. The method of claim 1, wherein step (a) comprises applying light energy to the plant surface.

3. The method of claim 1, wherein the plant is a tree, a vine, a forage, a perennial crop, a row crop, a bush crop, an ornamental plant, an annual plant, or a grass.

4. The method of claim 3, wherein the tree is a fruit tree selected from the group consisting of an apple tree, a peach tree, a citrus tree, an olive tree, a cherry tree, a pear tree, a plum tree, a grapefruit tree, and an apricot tree.

5. The method of claim 3, wherein the plant is a citrus tree.

6. The method of claim 5, wherein the citrus tree is selected from the group consisting of Citrus x sinesis, Citrus macrophylla, Citrus assamensis, Citrus aurantiaca, Citrus aurantiifolia, Citrus aurantium L., Citrus australasica, Citrus australis, Citrus cavaleriei, Citrus garrawayi, Citrus glauca, Citrus gracilis, Citrus halimii, Citrus hystrix, Citrus indica, Citrus inodora, Citrus japonica, Citrus junos, Citrus khasya, Citrus latifolia, Citrus latipes, Citrus limon (L.), Citrus maxima (Burm.), Citrus medica L., Citrus paradise, Citrus polyandra, Citrus x polytrifolia, Citrus reticulate, Citrus swinglei, Citrus trifoliata L., Citrus warburgina, and Citrus wintersii.

7. The method of claim 1, wherein the plant surface is a leaf surface.

8. The method of claim 1, wherein the substance is an exogenous nucleic acid, a fertilizer, an insecticide, a fungicide, a microelement, and a plant hormone.

9. The method of claim 8, wherein the substance is an exogenous nucleic acid.

10. The method of claim 9, wherein the exogenous nucleic acid is not incorporated in a viral vector.

11. The method of claim 9, wherein the exogenous nucleic acid is selected from the group consisting of an antisense RNA (asRNA), a micro RNA (miRNA), a small interfering RNA (siRNA), a double-stranded RNA (dsRNA), a non-coding RNA (ncRNA), mitochondrial RNA (mtRNA), and combinations thereof.

12. The method of claim 8, wherein the substance is a fertilizer selected from the group consisting of ammonium nitrate, Ammonium sulfate, anhydrous ammonia, calcium nitrate/urea, oxamide, potassium nitrate, urea, urea sulfate, ammoniated superphosphate, diammonium phosphate, nitric phosphate, potassium carbonate, potassium metaphosphate, calcium chloride, magnesium ammonium phosphate, magnesium sulfate, ammonium sulfate, and potassium sulfate, or a combination of any of the foregoing.

13. The method of claim 8, wherein the substance is an insecticide selected from the group consisting of Aldicarb, Bendiocarb, Carbofuran, Ethienocarb, Fenobucarb, Oxamyl, Methomyl, Acetamiprid, Clothianidin, Dinotefuran, Imidacloprid, Nitenpyram, Nithiazine, Thiacloprid, Thiamethoxam, Mirex, Tetradifon, Phenthoate, Phorate, Pirimiphos-methyl, Quinalphos, Terbufos, Tribufos, Trichlorfon, Tralomethrin, Transfluthrin, Fenoxycarb, Fipronil, Hydramethylnon, Indoxacarb, Limonene, Carbaryl, Propoxur, Endosulfan, Endrin, Heptachlor, Kepone, Lindane, Methoxychlor, Toxaphene, Parathion, Parathion-methyl, Phosalone, Phosmet, Phoxim, Temefos, Tebupirimfos, and Tetrachlorvinphos, or a combination of any of the foregoing.

14. The method of claim 8, wherein the substance is a fungicide selected from the group consisting of azoxystrobin, cyazofamid, dimethirimol, fludioxonil, kresoxim-methyl, fosetyl-A1, triadimenol, tebuconazole, and flutolanil, or a combination of any of the foregoing.

15. The method of claim 8, wherein the substance is a microelement is selected from the group consisting of iron, manganese, boron, zinc, copper, molybdenum, chlorine, sodium, cobalt, silicon, and nickel, or a combination of any of the foregoing.

16. The method of claim 8, wherein the substance is a hormone selected from the group consisting of heteroauxin, indolylbutyric acid, naphthylacetic acid, a gibberellin, a cytokinin, strigolactones, melatonin, and abscisic acid, or a combination of any of the foregoing.

17. The method of claim 1, wherein step (b) comprises brushing or spraying the substance onto the plant.
